(19) United States
(12) Patent Application Publication
(10) Pub. No.: US 2014/0309620 A1
(43) Pub. Date: Oct. 16, 2014

(54) TRANSILLUMINATING OBTURATOR

(71) Applicant: University of Iowa Research Foundation, Iowa City, IA (US)

(72) Inventor: Henry T. Hoffman, Iowa City, IA (US)

(73) Assignee: UNIVERSITY OF IOWA RESEARCH FOUNDATION, Iowa City, IA (US)

(21) Appl. No.: 14/251,032

(22) Filed: Apr. 11, 2014

Related U.S. Application Data
(60) Provisional application No. 61/810,856, filed on Apr. 11, 2013.

(51) Int. Cl. A61B 17/34

(52) U.S. Cl. CPC A61B 17/3403
USPC 604/506; 604/164.01; 604/165.04

(57) ABSTRACT

Methods and devices for placement of a medical device using illumination are disclosed.

### SUMMARY

Methods and devices for placement of a medical device using illumination are disclosed.

We describe the technique of transillumination to direct placement and confirm location of needle/trocar assemblies into the larynx. This approach exploits the air-filled nature of the laryngo-pharynx to permit visualization of mucosal surfaces overlying small solid anatomic regions. Specific sites targeted for therapy directed by this transillumination technique include the vocal folds, paraglottic space, and posterior larynx. Directed injection to the vocal folds by percutaneous puncture with a needle employing a transilluminating cable permits endoscopic view of the larynx via transnasal fiberoptics or via transoral laryngoscopy to confirm needle location. Application of this technique improves accuracy for injection to the vocal folds for augmentation as well as to the muscles for chemodenervation (including the posterior crico-arytenoid muscle). This technique is used to direct and confirm appropriate placement of the arytenoid repositioning device (ARD). The ARD is described in U.S. patent application Ser. No. 13/201,669, filed Aug. 16, 2011 (published as U.S.2011/0301580) which is hereby incorporated herein by reference in its entirety.

Transillumination is a commonly used technique in healthcare whereby the light is visualized as it shines through a structure in order to confirm the location of an instrument with a lighted tip. For example, the location of the tip of a sialendoscope is confirmed as deep within the parotid gland in FIG. 1.

Use of transillumination to help direct interventions has been common medical practice for many decades. For example, initial placement of feeding tubes through the abdominal wall were facilitated by placement of lighted endoscopes transorally into the stomach was developed in the 1970's by Gauderer and others. Gauderer' writes about percutaneous gastrostomies (PEG's) in a review about this early work that “gastric transillumiation and digital soma site identification, key components of the procedure, proved invaluable ”

Common medical applications of transillumination to localize instrumentation within the body have addressed cavities naturally filled with air (trachea/paranasal sinuses), gas insufflated cavities (abdomen) and fluid filled joint spaces. Also, intubation of the trachea can be facilitated by placing a light wand (flexible lighted stylet) through an endodtracheal tube confirming appropriate intubation by transilluminating through the neck skin.

### BRIEF DESCRIPTION OF THE DRAWINGS

FIG. 1 is a photograph showing a 1.6 mm diameter sialendoscope being placed into the left parotid duct orifice with location of the tip of the endoscope confirmed deep into the tail of the parotid gland by transillumination.

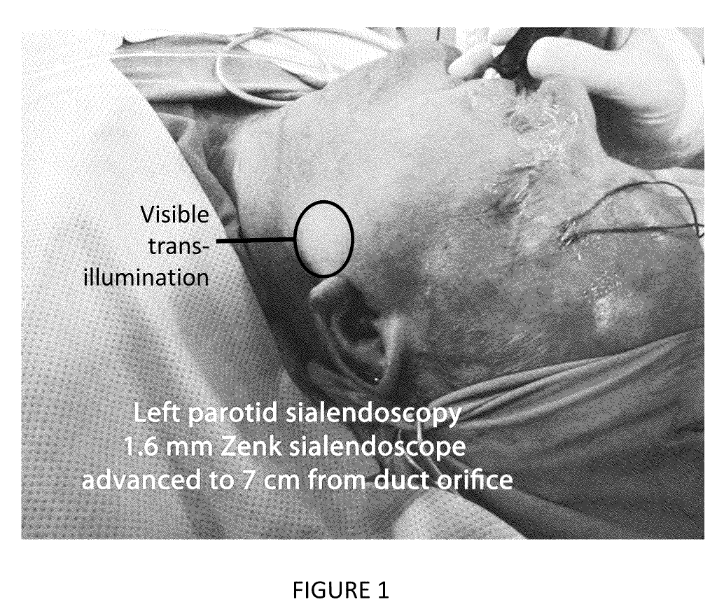

FIG. 2 is a photograph of a cadaveric human larynx with a trocar positioned through the paraglottic space via anterior fenestration in thyroid cartilage to engage the muscular process of the arytenoid. A light source (sialendoscope in this case) placed through the trocar confirms appropriate location by transilluminating through the mucosa of the deep in medial wall of the pyriform sinus.

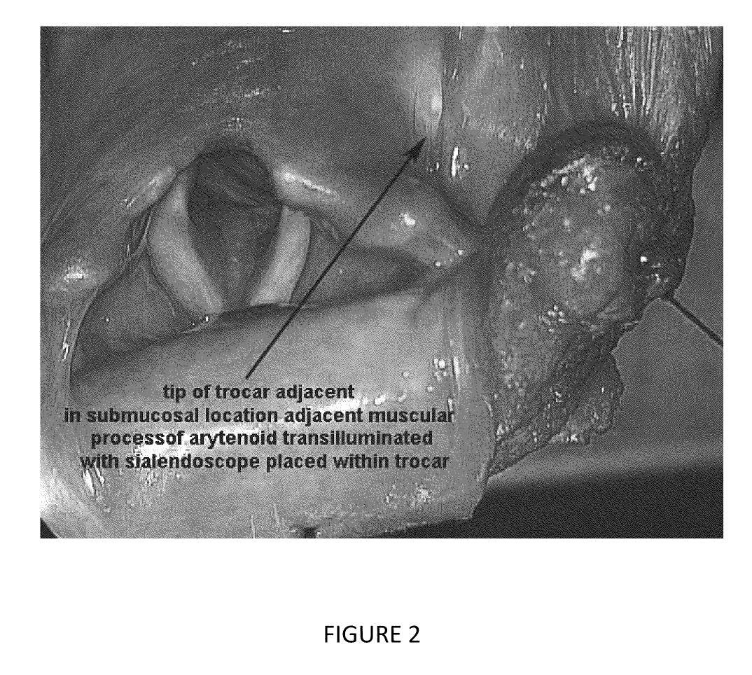

FIG. 3 is a photograph of an arytenoid repositioning device designed to be deployed through an 18-gauge trocar, a stylet or obturator sized to fill the lumen of an 18-gauge trocar, and the 18-gauge trocar itself.

FIG. 4 shows schematically a transilluminating obturator.

FIG. 5 shows schematically one embodiment of the distal tip of a transilluminating obturator and trocar.

### DETAILED DESCRIPTION

Trocars are hollow tubes inserted into anatomic regions in order to instill medications, to place instrumentation, and to inspect the internal structure. Obturators are solid rods that fit snugly inside the trocar to fill its tip in order to facilitate atraumatic introduction of the assembly into a body cavity cavity. Without an obturator, a trocar advanced through tissue will naturally collect a cored cylinder of tissue (core biopsy). The presence of the obturator inside the trocar blocks tissue from entering the hollow lumen of the trocar as the trocar is advanced. Once the trocar has been placed through a structure (skin or other), the obturator is removed, permitting the user to access the hollow inner tube of the trocar, for example for inspection, instillation, or placement of instrumentation. An example of this type assembly is a spinal needle whose obturator is secured within the hollow needle in order to minimize damage to the skin, muscles, ligament, and dura as the assembly is placed percutaneously into the cerebrospinal fluid (CSF) around the spinal cord. Once in position, the obturator is removed to permit use of the hollow needle to withdraw CSF, measure pressures of the CSF, or instill materials into the CSF.

We introduce the use of a modified obdurator that contains fiberoptics to serve the dual functions of (1) filling the hollow rod of a trocar (or needle) to facilitate passage into an anatomic structure and (2) directing placement of the trocar employing transillumation through fiberoptics within the obturator.

The use of fiberoptic guidance (transillumation) through a trocar or needle may be accomplished in the course of placing the assembly through adaptation of the obturator to incorporate the light source. Alternatively, a standard solid, non-illuminating obturator may guide initial placement of the trocar/needle and then can be followed by placement of a light source to confirm appropriate tip location of the needle/trocar.

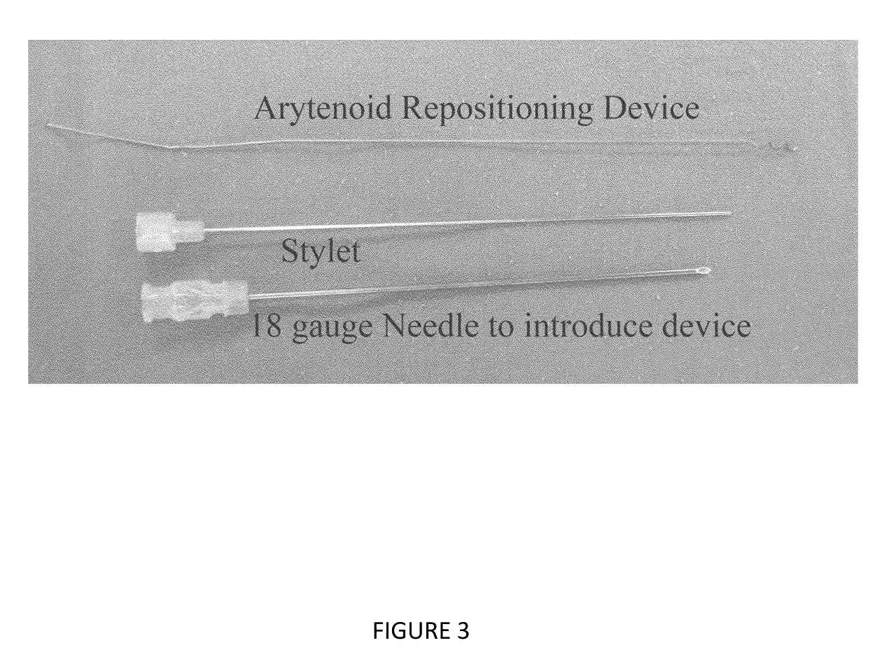

FIG. 3 shows an example of a kit including a trocar (or needle), an obturator (or stylet), and a device designed to be deployed through the trocar. In use, the obturator would be inserted into the trocar to block the internal lumen of the trocar. The plastic collar at the proximal end of the obturator will seat on and lock into the plastic collar at the proximal end of the trocar. In this example, the obturator is somewhat longer than the trocar so that, when the distal tips of the trocar and obturator are flush, the proximal end of the obturator extends beyond the proximal end of the trocar. A wide variety of alternative parts can serve the same purpose or function as the interlocking plastic collars shown in FIG. 3.

The affixed obturator/trocar assembly can then be advanced into the tissue. Once the distal tip of the trocar is correctly placed, the plastic collars can be disengaged and the obturator removed while the trocar is held in place. The trocar can be secured in place in the patient either by hand, or by mechanical means. Once the trocar is empty and correctly placed, the user can insert an instrument, medicaments, etc., through the lumen.

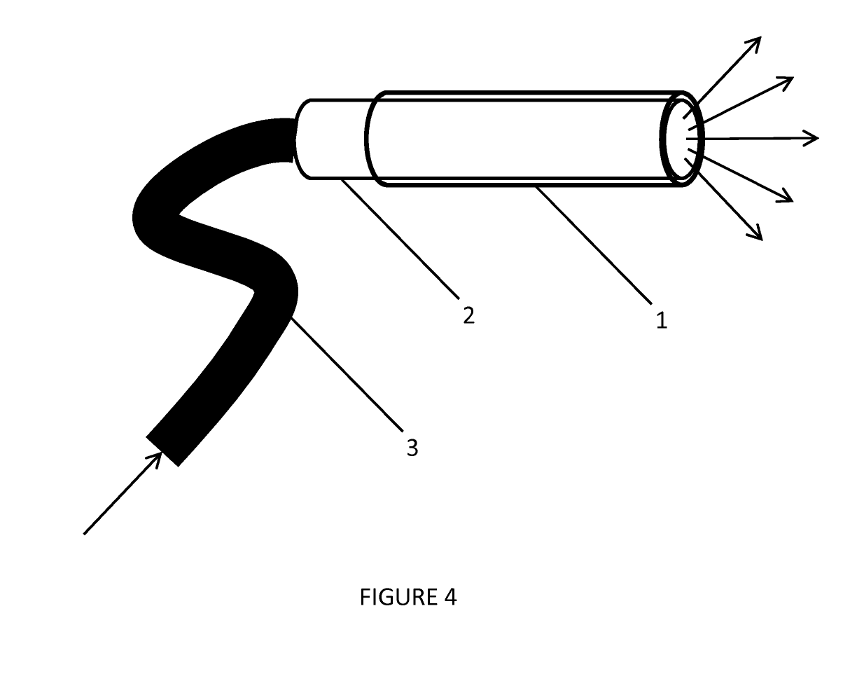

FIG. 4 shows schematically a transilluminating obturator. A trocar 1 defines a hollow lumen. An obturator 2 is inserted into the lumen of the trocar 1. A waveguide 3 is attached to the proximal end of the obturator 2. Light (shown as arrows) directed into the waveguide 3 is directed into the obturator 2 at the proximal end and out the distal end. The obturator 2 acts as a waveguide or in some other way functions to direct light entering its proximal end out its distal end.

In use, the obturator 2 is secured in place inside the trocar 1. The waveguide 3 is operably attached to a light source that directs light into the waveguide 3. The user then visualizes the area where he intends to deploy the distal tip of the trocar 1, for example, with an appropriate endoscopic device. The method of visualization should be sensitive to the wavelength of light provided by the light source. The obturator/trocar assembly is then inserted into the tissue as described above with the light source activated and the distal tip of the obturator/trocar illuminated. The user will be able to see, by way of the visualization, where the distal tip is because of the light escaping the distal end of the obturator, even if the user's view of the desired location of the tip is obstructed, so long as the tissue wall obstructing direct view is sufficiently translucent and the tip is sufficiently illuminated to allow visualization of the tip through the intervening tissue. In this way the user will be able to direct the distal tip to a desired location, even if the location cannot be directly visualized. The user can then be confident that the trocar has been correctly placed in a location that is difficult or impossible to directly view.

This method of transillumination to place a small trocar can be useful in a wide variety of contexts and body cavities. It may be particularly useful in the neighborhood of the larynx and vocal cords. Needles/trocars are often used in this part of the anatomy to deliver therapeutic injections. Filler substances such as collagen or Radiesse™ Voice are typically injected directly into the vocal cord to the deep vocal ligament. Biologically active substances such as hyaluronic acid or stem cells can be injected in the Reinke's space between the vocal ligament and the vocal cord epithelium. Neurotoxins such as Botox™ can be injected into muscles in the vocal cord (e.g., LCA, TA) or into muscles controlling vocal cord movement (PCA). In all these cases, but especially in the case of neurotoxins, it is important to make sure that the medicament is deployed in precisely the intended location. To achieve this benefit, it is important that the method of visualization provide the user with a sufficiently detailed view to distinguish specific subsites within the vocal cords and the larynx.

This method of transillumination can also be useful in procedures that employ expansion or suffusion of a cavity via a needle, in particular, microendoscopy of Reinkes Space. A correctly placed needle or trocar can supply an infusion of saline or air to expand such a cavity. The needle can also be followed by dilators.

This method of transillumination could also be useful in deploying a device such as the arytenoid repositioning device shown in FIG. 3.

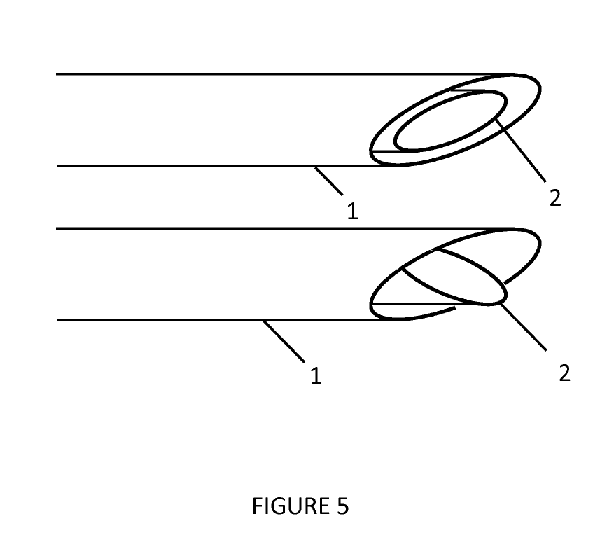

As shown in FIG. 4, the distal tips of the obturator and trocar lie entirely in a plane perpendicular to the longitudinal axes of the obturator and trocar. But some trocars and needles will include an angled end as shown in FIG. 5. For clarity, a radial gap is left between the trocar 1 and obturator 2 are shown in FIG. 5. In practice, the obturator 2 will typically substantially fill the trocar 1 in the radial direction. FIG. 5 shows the trocar 1 and obturator 2 in two different relative orientations. Above, the the trocar 1 and obturator 2 are oriented relative to each other such that their angled ends are substantially coplanar. This may be a desired orientation during insertion so that the obturator 2 properly and completely fills the end of the trocar 1. Below, the trocar 1 and obturator 2 are shown with the obturator 2 rotated 180 degrees about the obturator axis relative to the trocar 1. Orienting the trocar 1 and obturator 2 so that their ends are not perfectly aligned can be useful, for example, in adjusting the amount of illumination provided through the obturator 2. The proximal ends are not shown, but the trocar 1 and obturator 2 may include at their proximal ends markings that align when the trocar 1 and obturator 2 are oriented relative to each other such that their angled ends are substantially coplanar. The relative positions of the marks can then be used to indicate how aligned the angled distal ends of the trocar 1 and obturator 2 are.

Certain Embodiments

A kit can include a trocar, an obturator and a waveguide. The trocar can have a proximal end, a distal end, and a trocar axis between the ends. The trocar can define a lumen between its proximal and distal ends. The distal end of the trocar can be sharpened. The obturator can have a proximal end, a distal end, and an obturator axis between the ends. The length of the obturator from its proximal to its distal end can be greater than the length of the trocar from its proximal to its distal end. The obturator can also be configured to transmit light of a predetermined range of wavelengths from its proximal end to its distal end. The obturator can be sized and shaped to substantially fill the trocar in directions radial to the obturator and trocar axes when the obturator is disposed inside the trocar lumen with the obturator and trocar axes aligned. The proximal end of the obturator being configured to be operably connected to the waveguide.

The waveguide can be a fiber optic cable. The predetermined range of wavelengths can be in the range of 300 nanometers to 2000 nanometers, or any subset of that range. The kit can also include a light source capable of being operably connected to the waveguide. The light source can be a laser, incandescent light, fluorescent light, light emitting diode, or any other type of light source capable of emitting light having a wavelength in the predetermined wavelength range.

The obturator can include a first locking element adjacent to its proximal end. The trocar can include a second locking element adjacent to its proximal end. The first and second locking elements can be complementary to one another such that the first and second locking elements can reversibly transition between (a) a locked state in which the obturator and the trocar are fixedly attached to one another and (b) an unlocked state in which the obturator is free to slide into or out of the trocar and rotate relative to the trocar.

The sharpened distal end of the trocar can be configured to lie in a plane that is not perpendicular to the trocar axis; such a plane may have a particular orientation relative to the trocar axis. Similarly, the distal end of the obturator can lie in a plane that is not perpendicular to the obturator axis; such a plane may also have a particular orientation relative to the obturator axis. The two planes may or may not have the same orientation relative to their respective axes. The proximal end of the obturator can include an obturator mark disposed so as to visible to a user when the obturator is disposed inside the trocar with the distal end of the obturator flush with the distal end of the trocar. Likewise, the proximal end of the trocar can include a trocar mark disposed so as to visible to a user when the obturator is disposed inside the trocar with the distal end of the obturator flush with the distal end of the trocar. The obturator mark and trocar mark can be disposed such that, when the obturator mark is aligned with the trocar mark, the distal end of the obturator and the distal end of the trocar are substantially co-planar.

A device can include the elements of any such kit, with the waveguide operably connected to the proximal end of the obturator, and the obturator positioned substantially inside the lumen of the trocar.

A method of using such a kit can include: (a) operably connecting the waveguide to a light source and to the proximal end of the obturator, the light source providing light having wavelengths within the predetermined range of wavelengths such that is emitted from the light source and emerges from the distal end of the obturator; (b) inserting the obturator into the lumen of the trocar so that the obturator substantially fills the lumen and so that the distal ends of the trocar and the obturator are substantially flush; (c) visualizing the desired location in the patient; (d) inserting the trocar into the patient without moving the obturator relative to the trocar; (e) based on the visualization of the desired location in the patient, determining whether the distal tip of the trocar is in the desired location; (f) if the distal tip of the trocar is not in the desired location, repeating steps (d) and (e) until the distal tip of the trocar is in the desired location; (g) securing the trocar in place so that the distal tip of the trocar cannot substantially move from the desired location; and (h) removing the obturator from the trocar.

Where the kit includes the above-described obturator mark and trocar mark, the method can also include rotating the obturator about the obturator axis with respect to the trocar without translating the obturator with respect to the trocar so that the obturator mark and the trocar mark occupy a predetermined desired relative position. That predetermined desired relative position can be alignment of the marks with one another, anti-alignment, or anything in between in any direction.

Such methods can further include injecting a medicament through the trocar. Such methods can also or alternatively include inserting a device, such as an arytenoid repositioning device, through the trocar.

I claim:

1. A kit comprising a trocar, an obturator and a waveguide, wherein:
the trocar has a proximal end, a distal end, and a trocar axis between the ends, the trocar defining a lumen between its proximal and distal ends, the distal end of the trocar being sharpened;
the obturator having a proximal end, a distal end, and an obturator axis between the ends, the length of the obturator from its proximal to its distal end being greater than the length of the trocar from its proximal to its distal end, the obturator also being configured to transmit light of a predetermined range of wavelengths from its proximal end to its distal end, the obturator being sized and shaped to substantially fill the trocar in directions radial to the obturator and trocar axes when the obturator is disposed inside the trocar lumen with the obturator and trocar axes aligned; and
the proximal end of the obturator being configured to be operably connected to the waveguide.

2. The kit of claim 1 wherein the waveguide is a fiber optic cable.

3. The kit of claim 1 wherein the predetermined range of wavelengths is in the range of 300 nanometers to 2000 nanometers.

4. The kit of claim 1 wherein:
the obturator comprises a first locking element adjacent to the proximal end of the obturator;
the trocar comprises a second locking element adjacent to the proximal end of the trocar; and
the first and second locking elements being complementary to one another such that the first and second locking elements can reversibly transition between (a) a locked state in which the obturator and the trocar are fixedly attached to one another and (b) an unlocked state in which the obturator is free to slide into or out of the trocar and rotate relative to the trocar.

5. The kit of claim 1 further comprising a light source capable of being operably connected to the waveguide.

6. The kit of claim 1 wherein the light source is a laser.

7. The kit of claim 1 wherein the sharpened distal end of the trocar lies in a plane that is not perpendicular to the trocar axis.

8. The kit of claim 1 wherein the distal end of the obturator lies in a plane that is not perpendicular to the obturator axis.

9. The kit of claim 8 wherein the sharpened distal end of the trocar lies in a plane that is not perpendicular to the trocar axis.

10. The kit of claim 9 wherein:
the proximal end of the obturator includes an obturator mark disposed so as to visible to a user when the obturator is disposed inside the trocar with the distal end of the obturator flush with the distal end of the trocar;
the proximal end of the trocar includes an trocar mark disposed so as to visible to a user when the obturator is disposed inside the trocar with the distal end of the obturator flush with the distal end of the trocar; and
the obturator mark is aligned with the trocar mark when the distal end of the obturator and the distal end of the trocar are substantially co-planar.

11. A device comprising the kit of claim 1 wherein:
the obturator is positioned substantially inside the lumen with the distal end of the obturator flush with the distal end of the trocar; and
the proximal end of the obturator is operably connected to the waveguide.

12. A method of using the kit of claim 1 to place the distal tip of the trocar at a desired location in a patient, the method comprising:
(a) operably connecting the waveguide to a light source and to the proximal end of the obturator, the light source providing light having wavelengths within the predetermined range of wavelengths such that is emitted from the light source and emerges from the distal end of the obturator;
(b) inserting the obturator into the lumen of the trocar so that the obturator substantially fills the lumen and so that the distal ends of the trocar and the obturator are substantially flush;
(c) visualizing the desired location in the patient;
(d) inserting the trocar into the patient without moving the obturator relative to the trocar;
(e) based on the visualization of the desired location in the patient, determining whether the distal tip of the trocar is in the desired location;
(f) if the distal tip of the trocar is not in the desired location, repeating steps (d) and (e) until the distal tip of the trocar is in the desired location;
(g) securing the trocar in place so that the distal tip of the trocar cannot substantially move from the desired location; and
(h) removing the obturator from the trocar.

13. A method of using the kit of claim 10 to place the distal tip of the trocar at a desired location in a patient, the method comprising:
(a) operably connecting the waveguide to a light source and to the proximal end of the obturator, the light source providing light having wavelengths within the predetermined range of wavelengths such that is emitted from the light source and emerges from the distal end of the obturator;
(b) inserting the obturator into the lumen of the trocar so that the obturator substantially fills the lumen and so that the distal ends of the trocar and the obturator are substantially flush;
(c) visualizing the desired location in the patient;
(d) inserting the trocar into the patient without moving the obturator relative to the trocar;
(e) rotating the obturator about the obturator axis with respect to the trocar without translating the obturator with respect to the trocar so that the obturator mark and the trocar mark occupy a predetermined desired relative position;
(f) based on the visualization of the desired location in the patient, determining whether the distal tip of the trocar is in the desired location;
(g) if the distal tip of the trocar is not in the desired location, repeating steps (d) and (e) until the distal tip of the trocar is in the desired location;
(h) securing the trocar in place so that the distal tip of the trocar cannot substantially move from the desired location; and
(i) removing the obturator from the trocar.

14. The method of claim 12 further comprising (i) inserting an arytenoid repositioning device through the trocar.

15. The method of claim 12 further comprising (i) injecting a medicament through the trocar.
